(19) United States
(12) Patent Application Publication
(10) Pub. No.: US 2015/0056702 A1
(43) Pub. Date: Feb. 26, 2015

(54) METHOD AND KIT FOR CULTURING STEM CELLS

(71) Applicant: China Medical University, Taichung City (TW)

(72) Inventors: SHIH-PING LIU, TAICHUNG CITY (TW); SHINN-ZONG LIN, TAICHUNG CITY (TW); HORNG-JYH HARN, TAICHUNG CITY (TW); YING-JIUN CHIEN, TAICHUNG CITY (TW); CHIEN-YU HSU, TAICHUNG CITY (TW); CHENG-HSUAN CHANG, TAICHUNG CITY (TW)

(21) Appl. No.: 14/529,808

(22) Filed: Oct. 31, 2014

Related U.S. Application Data
(62) Division of application No. 13/826,065, filed on Mar. 14, 2013.

(30) Foreign Application Priority Data
Jan. 25, 2013 (TW) 102102807

(51) Int. Cl. C12N 5/00

(52) U.S. Cl. CPC C12N 5/0018; C12N 2501/40; C12N 2500/30
USPC 435/383; 435/404

(57) ABSTRACT

A method is provided, including adding a phthalide to a stem cell medium to provide a phthalide-containing medium, and then culturing a stem cell using the phthalide-containing medium. The use of a phthalide in a medium for culturing stem cells optionally maintains the pluripotency of stem cells. The phthalide also enhances the generation efficiency of induced pluripotent stem cells to decrease the culture cost.

This application claims priority to Taiwan Patent Application No. 102102807 filed on Jan. 25, 2013, in the Taiwan Intellectual Property Office, the disclosure of which is incorporated herein in its entirety by reference.

### CROSS-REFERENCES TO RELATED APPLICATIONS

Not applicable.

### BACKGROUND

1. Field of the Invention

The present invention relates to a method for culturing a stem cell, particularly relates to the use of a phthalide in culturing a stem cell; the present invention also relates to a kit for culturing a stem cell, wherein the kit comprises a phthalide.

2. Descriptions of the Related Art

The medical technology to date is still deficient in effective therapeutic methods for many diseases, such as diabetes mellitus, severe anemia, apoplexy, Alzheimer's disease, amyotrophic lateral sclerosis, and Parkinson's disease. The pluripotency of stem cells brings a ray of light to the patients suffering from these diseases.

Stem cells, depending on their ability to self-renew and differentiate, can be classified into the following four types: totipotent stem cells, pluripotent stem cells, multipotent stem cells, and unipotent stem cells. On the other hand, depending on the appearance order during the developmental process and distributional profile of stem cells, stem cells can be classified into the following two types: embryonic stem cells (ES cells) and adult stem cells. Both ES cells and induced pluripotent stem cells (iPS cells) are typical stem cells, which are capable of differentiating into three embryonic germ layers, including an endodermal layer, a mesodermal layer and an ectodermal layer. These stem cells have a high self-renewal efficiency, and thus are of the most developmental potential.

ES cells can be effectively cultured into specialized cells, such as cardiomyocytes, hepatocytes, pancreatic cells or ova, and thus can be used for the transplantation of cells or organs. In other aspects, the source of human ES cells (such as remaining embryos obtained from infertility treatment, embryonic primordial germ cells obtained from abortion, and fused cells) is still controversial. Therefore, research has been conducted to induce cell reprogramming by introducing specific genes into matured fibroblast cells (obtained from the patient's skin) so as to form cells with characteristics and functions similar to those of an embryonic stem cell (i.e., to form an induced pluripotent stem cells), and then, successfully make these cells differentiate into organ(s) of a human body without immunological rejection.

The term “pluripotent stem cells” used in this application refers to stem cells with a characteristic of incomplete differentiation, as well as an ability to proliferate and differentiate into different cell types to form various mature tissues. The pluripotency of stem cells is an essential factor for the research of disease and the therapeutic application, and therefore, it is a great topic to study how to culture stem cells while maintaining their pluripotency. Currently, it is general to use a multifunctional cell factor, i.e., leukemia inhibitory factor (LIF) which has been found in the late 1960s to maintain the pluripotency of stem cells. However, the cost for culturing stem cells is high due to the high price of LIF. In addition, although iPS cells have great potential for clinical application, its application is limited due to generation inefficiency.

In view of the above issues about culturing stem cells, there is still a need for an effective method for culturing stem cells to enhance the applicability of stem cells. The inventors of the present invention found that a phthalide can effectively maintain the pluripotency of stem cells, and thus, can be used as a substitution for LIF to reduce the cost of culturing stem cells. The inventors also found that the phthalide can partially solve the problem of generation inefficiency, thereby, enhancing the applicability of stem cells as well.

### SUMMARY

An objective of the present invention is to provide a method for culturing a stem cell, comprising:
(A) providing a stem cell medium;
(B) adding a phthalide into the stem cell medium to provide a phthalide-containing medium; and
(C) using the phthalide-containing medium to culture a stem cell.

Another objective of this invention is to provide a use of a phthalide in culturing a stem cell.

Yet a further objective of this invention is to provide a kit for culturing a stem cell, comprising (1) a stem cell medium; and (2) a phthalide.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

The patent application contains at least one drawing executed in color. Copies of this patent document with color drawing(s) will be provided by the Office upon request and payment of the necessary fee.

FIG. 1 is a statistical bar diagram showing the survival rate of ES cells after culturing;

FIG. 2 is a statistical bar diagram showing gene expression levels of Oct4 and Sox2 in ES cells after culturing (*p<0.05: statistical significance);

FIG. 3 is a staining picture (left) and a statistical bar diagram (right) showing the expression level of alkaline phosphatasde (AP) in ES cells after culturing (*p<0.05: statistical significance);

FIG. 4 is a staining picture (left) and a statistical bar diagram (right) showing the expression level of alkaline phosphatasde (AP) in iPS cells after culturing (*p<0.05: statistical significance);

FIGS. 5A to 5D are immunofluorescence staining pictures (FIG. 5A and FIG. 5C) and statistical bar diagrams (FIG. 5B and FIG. 5D) showing the protein expression levels of Nanog and SSEA1 in ES cells after culturing (*p<0.05: statistical significance);

FIGS. 6A to 6D are immunofluorescence staining pictures (FIG. 6A and FIG. 6C) and statistical bar diagrams (FIG. 6B and FIG. 6D) showing the protein expression levels of Nanog and SSEA1 in iPS cells after culturing (*p<0.05: statistical significance);

FIG. 7 is an immunofluorescence staining picture showing the three embryonic germ layers of ES cells after culturing with a phthalide-containing medium;

FIG. 8 is a statistical bar diagram showing the mRNA expression levels of Jak2 and Stat3 gene in ES cells after culturing (*p<0.05: statistical significance);

FIG. 9 is a Western blot picture (left) and a statistical bar diagram (right) showing the expression levels of phosphorylated-Jak2 and phosphorylated-Stat3 in ES cells after culturing (*p<0.05: statistical significance);

FIG. 10 is a statistical bar diagram showing the mRNA expression levels of LIF, EGF, IL5, IL11, EPO and OSM gene in ES cells after culturing (*p<0.05: statistical significance);

FIG. 11 is a statistical bar diagram showing the GFP fluorescence intensity in the iPS cells formed after culturing (*p<0.05: statistical significance);

FIG. 12 is an immunofluorescence staining picture showing the iPS cells formed after culturing; and

FIG. 13 is an immunofluorescence staining picture showing the three embryonic germ layers of iPS cells formed after culturing with a phthalide-containing medium.

### DETAILED DESCRIPTION

The following will describe some embodiments of the present invention in detail. However, without departing from the spirit of the present invention, the present invention may be embodied in various embodiments and should not be limited to the embodiments described in the specification. In addition, unless otherwise state herein, the expressions “a,” “the,” or the like recited in the specification of the present invention (especially in the claims) should include both the singular and plural forms.

The present invention provides a method for culturing a stem cell, which is characterized by the addition of a phthalide into a stem cell medium. The present invention comprises the following steps: (A) providing a stem cell medium; (B) adding a phthalide into the stem cell medium to provide a phthalide-containing medium; and (C) using the phthalide-containing medium to culture a stem cell.

The stem cell medium used in step (A) of the method of the present invention refers to a medium containing essential nutrients and conditions (e.g. pH) for the growth and differentiation of stem cells. The components of the stem cell medium may be adjusted depending on the types of the stem cells to be cultured (i.e. the stem cells in the step (C)). In general, a stem cell medium comprises a base culture medium, an animal serum (e.g. fetal bovine serum), non-essential amino acids (NEAA) and L-glutamine, etc. Examples of the base medium suitable for the method of the present invention include, but are not limited to, DMEM (Dulbecco's Modified Eagle's Medium), MEM (Minimum Essential Medium), α-MEM (α-Minimum Essential Medium), BME (Basal Media Eagle), MEM/F12 medium, Ham's F10 medium, Ham's F12 medium, and RPMI (Rosewell Park Memorial Institute). In one embodiment of the method of the present invention, the base medium is DMEM.

The inventors of the present invention found that the addition of a phthalide into the stem cell medium can maintain the pluripotency of the cultured stem cells and can also address the issue of generation inefficiency of stem cells. Therefore, in step (B) of the method of the present invention, a phthalide is added into the stem cell medium in step (A) to provide a phthalide-containing medium. It is preferable that the phthalide comprises one or more compounds with the following formula (I):

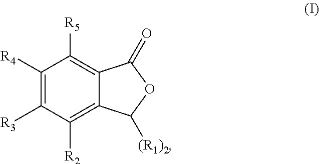

wherein

each R₁ is independently H, —OH, halogen, C₁-C₈ alkyl group, C₁-C₈ haloalkyl group, C₂-C₈ alkenyl group, phenyl group, naphthyl group or indolyl group, wherein, the phenyl group, naphthyl group and indolyl group are optionally substituted by —OH, halogen, amino group and/or C₁-C₄ alkyl group; and

R₂, R₃, R₄ and R₅ are independently H, —OH, halogen, cyano group, amine group, carboxyl group, Cβ1-C₈ alkyl group, C₁-C₈ haloalkyl group or C₁-C₈ alkoxy group;

with a proviso that not all of R₁, R₂, R₃, R₄ and R₅ are H.

Preferably, the phthalide used in the method of the present invention comprises, but are not limited to, one or more compounds selected from a group consisting of methylphthalide, 7-methylphthalide, ethylphthalide, propylidenephthalide, butylphthalide, n-butylidenephthalide, 3-bromophthalide, 5-bromophthalide, 5-chlorophthalide, 6-chlorophthalide, 3,4-dichlorophthalide, tetrachlorophthalide, 3-hydroxy-3-trifluoromethylphthalide, 3-methyl-3-(1-naphthyl)phthalide, 3-(5-fluoro-1-naphthyl)phthalide, 4-amino-3-hydroxyphthalide, 5-carboxyphthalide, 5-cyanophthalide, 7-methoxylphthalide, 7-hydroxy-6-methoxyphthalide, 3-(1,2-dimethyl-3-indolyl)phthalide and phenolphthalein.

More preferably, the phthalide used in the method of the present invention comprises one or more compounds selected from a group consisting of n-butylidenephthalide, butylphthalide, tetrachlorophthalide, and phenolphthalein. Most preferably, the phthalide is n-butylidenephthalide. N-butylidenephthalide, generally abbreviated as “BP” or “bdph,” can be purified and isolated from Chinese medicines or be obtained by a chemical synthesis method. In one embodiment of the present invention, BP is purified from a Chinese medicine, Angelica sinensis (the roots are usually used).

In step (B) of the method of the present invention, the phthalide may directly be added into the stem cell medium, and then dissolved into the stem cell medium to provide a phthalide-containing medium. Alternatively, the phthalide may be dissolved into a solvent to provide a phthalide-containing solution first, and then the phthalide-containing solution is mixed with the stem cell medium from step (A) to ensure the dissolution of phthalide to improve the usage efficiency of the phthalide. For example, when BP is used as the phthalide in step (B), step (B) may comprise (b1) dissolving BP into a solvent to form a phthalide-containing solution; and (b2) mixing the BP-containing solution with the stem cell medium obtained from step (A) (e.g. adding the phthalide-containing solution into the stem cell medium). The solvent used in the step (b1) is usually a polar solvent, such as dimethyl sulfoxide (DMSO) and ethanol.

In the method of the present invention, the amount of the phthalide used in step (B) may range from about 1 μg to about 80 μg, preferably from about 5 μg to about 50 μg, and more preferably from about 8 μg to about 12 μg per milliliter of the stem cell medium. For instance, as illustrated in the examples provided hereinafter, when BP is used as the phthalide to culture ES cells, an amount of BP ranging from about 9 μg to about 11 μg per milliliter of the stem cell medium could effectively maintain the pluripotency of ES cells. On the other hand, when BP is used to culture iPS cells, an amount of BP ranges from about 5 μg to about 10 μg per milliliter of the stem cell medium could effectively maintain the pluripotency of iPS cells and enhance the generation efficiency.

In step (C) of the method of the present invention, the phthalide-containing stem cell medium obtained from step (B) is used to culture stem cells. In principle, the method of the present invention can be used to culture any suitable stem cell, such as a cell selected from a group consisting of an ES cell, an iPS cell, a mesenchymal stem cell, an adipose stem cell, a hematopoietic stem cell, and combinations thereof. In some embodiments of the method of the present invention, the stem cell cultured in step (C) is selected from the following group: an ES cell, an iPS cell, and a combination thereof. The ES cells could be retrieved from the embryos of a mouse in the blastula stage. The iPS cells could be provided by co-transfecting four genes, Oct4, Sox2, c-Myc and KIF4, into mouse embryonic fibroblast cells.

Any suitable culturing conditions could be chosen and used in step (C) depending on the types of the cultured stem cells without any particular limitation. In general, step (C) comprises culturing the stem cells onto a layer of feeder cells under conditions of from about 35° C. to 39° C., from about 3% to 7% CO₂ and from about 90% to 99% humidity. A feeder cell is a common material used for culturing stem cells and familiar to persons skilled in this field, and thus will not be further described here.

Without being limited by theory, it is believed that phthalide maintains the pluripotency of stem cells (especially ES cells and iPS cells) by activating the Jak2-Stat3 signaling pathway, to avoid the decrease of the survival rate and enhance the generation efficiency of stem cells, especially iPS cells.

Accordingly, the present invention also relates to the use of a phthalide in culturing a stem cell. The properties and features of the phthalide and stem cells are all as described above. In addition, the functional pathway and applied model of the use are also as described above.

The present invention further provides a kit for culturing a stem cell, comprising (1) a stem cell medium and (2) a phthalide. The conditions and methods for using the stem cell medium and the phthalide are all as described above. In addition, the kit of the present invention can optionally include a solvent such as a polar solvent to dissolve the phthalide for the subsequent usage. Examples of the polar solvent includes, but is not limited to, DMSO and ethanol.

Components (1) and (2) of the kit of the present invention are packaged and stored separately, and could be transported, sold separately or in set. Component (1) is combined with component (2) at the customer's facility prior to use according to the planned culture procedure and processes. Optional components may be placed in either the first container for component (1), the second container for component (2) and/or a third container. For instance, a polar solvent for the phthalide may be placed in either the second container for component (2) and/or a third container.

According to the present invention, a phthalide was used to replace the expensive LIF to maintain the pluripotency of stem cells, enhance the generation efficiency of some stem cells, effectively improve the stem cell culture procedure, and improve the development of clinical medicine.

The present invention will be further illustrated in detail with specific examples as follows. However, the following examples are provided only for illustrating the present invention, and the scope of the present invention is not limited thereby.

### EXAMPLES

### Example 1

### Preparation of Feeder Cells

Primary mouse embryonic fibroblast (MEF) cells were isolated from the 13.5 d-old embryos of C57BL/6 mice. The embryos were retrieved by Cesarean section, and the heads, legs, internal organs and tails of the embryos were removed. The remaining embryo parts were minced with fine scissors and placed in a tube containing trypsin for cell digestion. A pre-warmed MEF medium [DMEM+10% heat-inactivated FBS+penicillin (100 U/ml)+streptomycin (100 U/ml)+NEAA (0.1 mM)+L-glutamine (2 mM)] was added to culture the MEF cells in an incubator (37° C., 5% CO₂) for 1 hour. Then, the MEF cells were cultured in a culture dish with a pre-warmed cell medium [DMEM+15% heat-inactivated FBS+NEAA (0.1 mM)+L-glutamine (2 mM)] in an incubator (37° C., 5% CO₂) for 2 hours. The cells were treated with mitomycin C (10 μg/ml) to inhibit their proliferation, thereby, providing feeder cells necessary for the culture of ES cells.

### Example 2

### Culture of Stem Cells

Experiment A. Culture of Embryonic Stem (ES) Cells (Experimental Group)

The following steps were conducted in order to culture ES cells:
(1) preparing a stem cell medium: DMEM+15% heat-inactivated FBS+NEAA (0.1 mM)+L-glutamine (2 mM)+β-mercaptoethanol (0.2 mM);
(2) preparing a BP-containing solution: dissolving BP into DMSO to form a BP-containing solution (100 mg/ml), and storing the solution at −20° C.;
(3) adding different amounts of the BP-containing solution in step (2) into the stem cell medium in step (1) to form different BP-containing mediums, wherein the amount of BP were about 5, 10, 20 or 40 μg per milliliter of the stem cell medium respectively (i.e. the experimental group mediums: BP5, BP10, BP20 and BP40); and
(4) using the BP-containing medium of in step (3) to culture a ES cell (retrieved from the embryos in the blastula stage of 129sv/J mice) at 37° C., 5% CO₂ and 95% humidity on the feeder cells provided by Example 1.

Experiment B. Culture of Induced Pluripotent Stem (iPS) Cells (Experimental Group)

The protocol shown in experiment A was repeated, while an iPS cell (received from Riken Research Center, Japan) was cultured at step (4).

Experiment C. Culture of ES Cells (Control Group)

The following procedure was conducted in proper order to culture the ES cells:
(1) preparing a stem cell medium:
   LIF-containing medium (without BP): DMEM+15% heat-inactivated FBS+NEAA (0.1 mM)+L-glutamine (2 mM)+(3-mercaptoethanol (0.2 mM)+LIF (4 ng/ml); and
   control medium (without BP): DMEM+15% heat-inactivated FBS+NEAA (0.1 mM)+L-glutamine (2 mM)+β-mercaptoethanol (0.2 mM);
(2) using the medium in step (1) to culture a ES cell (retrieved from embryos in the blastula stage of 129sv/J mice) at 37° C., 5% CO₂ and 95% humidity on the feeder cells provided by Example 1.

Experiment D. Culture of Induced iPS Cells (Control Group)

The protocol shown in experiment C was repeated, while an iPS cell (received from Riken Research Center, Japan) was cultured at step (2).

### Example 3

### Examination of Cell Survival (MTT Assay)

In this example, 3-(4,5-dimethylthiazol-2-yl)-2,5-diphenyl tetrazolium bromide (MTT) was used to determine if the cell survival rate of stem cells will be influenced when BP was served as a substitute for LIF.

MTT is a water-soluble tetrazolium salt which can react in the mitochondrial respiratory chain in living cells, to metabolize and reduce the tetrazolium bromide shown in the structure of MTT and to form an water-insoluble purple crystal formazan under the reaction of succinate dehydrogenase (SDH) and cytochrome c (cyt c). The amount of the produced crystal is directly proportional to the number of living cells (because the SDH will disappear from dead cells and the MTT cannot be reduced). Furthermore, mitochondrium is an organelle in cells most sensitive to the environment, and thus, the MTT assay could serve as a marker of the survival rate of cells treated by a drug.

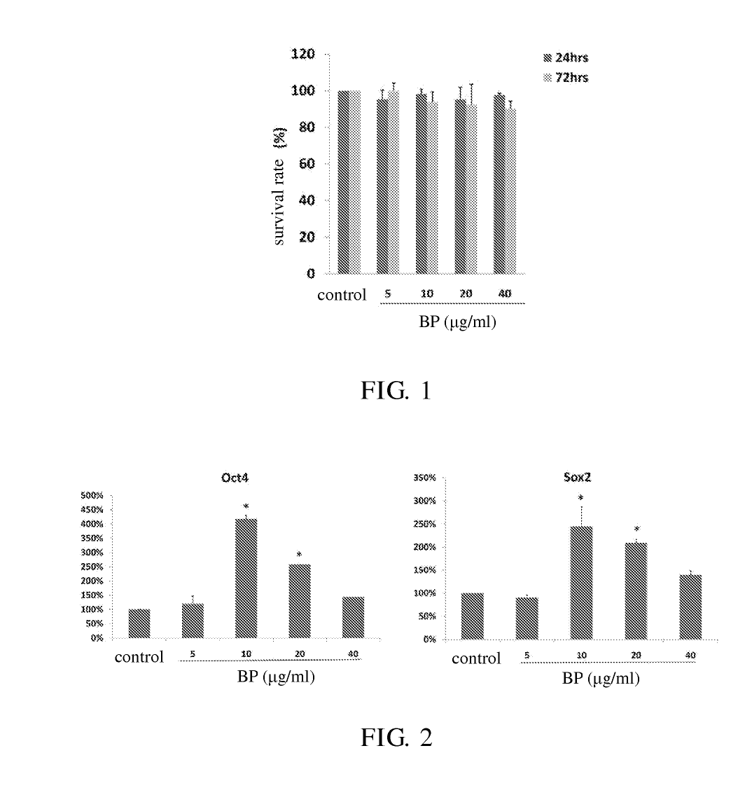

An experimental group medium (BP5, BP10, BP20 or BP40) and a control group medium (without BP) were added respectively into a 96-well micro-culture plate to culture ES cells with an initial density of 5×10³ per well for 24 to 72 hours. Then, 10 μl of 0.5 mg/ml MTT (Sigma) was added into each well and the cells were incubated in an incubator at 37° C. and 5% CO₂ for 2 to 4 hours. The medium was removed, and 100 μl of DMSO was added into each well and maintained at 37° C. for 10 minutes. The absorbances of the samples were measured at a wavelength of 570 nm to obtain the data of the experimental group (with BP) and control group (without BP). The absorbance of control group was served as a reference to calculate the relative cell survival rate of each experimental group. The results are shown in Table 1 and FIG. 1.

As shown in Table 1 and FIG. 1, the addition of BP into the ES cell medium will not affect the survival rate of stem cells.

TABLE 1

| | 24 hours | SD | 72 hours | SD |
|---|---|---|---|---|
| control | 100 | — | 100 | — |
| BP5 | 95.212 | 5.236 | 99.816 | 4.295 |
| BP10 | 98.092 | 2.782 | 93.848 | 5.425 |
| BP20 | 95.076 | 6.852 | 92.399 | 11.197 |
| BP40 | 97.550 | 1.060 | 90.128 | 4.219 |

### Example 4

### Expression Level of Stem Cell-Related Genes

Experiment I. Extraction of RNA

An experimental group medium (BP5, BP10, BP20 or BP40) and a control group medium (without BP) were added respectively to perform the following steps. ES cells were cultured with an initial density of 1×10⁴ per well in a 6-well culture dish. The medium was removed until the cells growth to be about 70 to 80% confluence, and then 1 ml TRIzol (Invitrogen) was added into each well. The cells were incubated for 5 minutes, and then was scraped by a spatula and placed into a 1.5 ml microtube to be lysed adequately. Chloroform (0.2 ml) was added into the microtubes. The mixture was shaken up and down for 15 seconds, and then incubated at room temperature for 2 to 3 minutes. Thereafter, the mixture was centrifuged at 12000 rpm at 4° C. for 15 minutes, and the supernatant was removed into another 1.5 ml microtube and isopropanol (0.5 ml) was added into the microtube with well mixing. The mixture was incubated at room temperature for 10 minutes and then centrifuged at 12000 rpm at 4° C. for 10 minutes. The supernatant was then removed. One ml of 70% ethanol containing DEPC.H₂O was used to wash the remaining sediment. The sediment was centrifuged at 7500 rpm at 4° C. for 5 minutes. The supernatant was removed. The sediment was dried by vacuum suction. The dried samples were dissolved in about 0.01 to 0.02 ml of DEPC.H₂O to obtain the experimental group RNA and control group RNA. Both groups were stored at −80° C.

Experiment II. Preparation of cDNA

The OD value of RNA samples in both groups obtained from the above Experiment I were measured at a wavelength of 260 nm (RNA: OD260=1 for a 40 μg/mL solution). Each RNA sample (2 μl) was added with RNA-free water until the volume of the sample reached 10 μl. Two μl oligo (dT) (100 μg/ml) was added into each sample, and the samples were incubated at 65° C. for 5 minutes. Then, the samples were immediately placed on ice and spun down. Next, 6.5 μl of a reaction solution [4 μl of 5× buffer+1 μl of 0.1 M DTT+1 μl of RNase out+0.5 μl of SSIII (Invitrogen)] was added into each of the samples, and the samples were incubated at 50° C. for about 30 to 60 minutes and at 75° C. for 15 minutes to obtain the experimental group cDNA and control group cDNA. Both groups were stored at −80° C.

Experiment III. Real-Time Polymerase Chain Reaction (Real-Time PCR; Q-PCR)

This experiment was used to detect the expression levels of ES cell-related genes, i.e., Oct4 and Sox2, etc. First, 4 μl of the experimental group cDNA or control group cDNA was added into each well of a 96-well micro-reaction plate, and mixed with 6 μl of a reaction solution [0.5 μl of 6 μM forward primer+0.5 μl of 6 μM reverse primer+5 μl of SYBR Green PCR Master mix (Roche)], wherein the SYBR Green PCR Master mix included the reagents needed for Q-PCR, such as dNTPs, MgCl, Taq DNA polymerase and 2×SYBR® Green I solution. As shown in Table 2, the primers were selected according to the gene to be measured. The mixture was mixed well, spun down, and placed into a real-time PCR machine (StepOnePlus™ Real-Time PCR System, Applied Biosystems). The reaction was performed at 95° C. for 10 minutes, and then conducted 40 cycles (reaction at 95° C. for 15 seconds and at 60° C. for 60 seconds). The cycle threshold (CT) values were measured. The results are shown in FIG. 2 and Table 3.

TABLE 2

| | | Nucleic |
|---|---|---|
| | | acid sequence |
| Name | | of primer |
| Oct4 | Forward primer | GAGGCTACAGGGACACCTTTC |
| | (SEQ ID No: 1) | |
| | Reverse primer | GTGCCAAAGTGGGGACCT |
| | (SEQ ID No: 2) | |
| Sox2 | Forward primer | AGGGCTGGACTGCGAACTG |
| | (SEQ ID No: 3) | |
| | Reverse primer | TTTGCACCCCTCCCAATTC |
| | (SEQ ID No: 4) | |

As shown in FIG. 2 and Table 3, the gene expression levels of Oct4 and Sox2 in the experimental group comprising the BP-containing ES cell culture medium are significantly up-regulated as compared with those of the control group. The group using BP10 has the most significant effect.

TABLE 3

| | Control group | BP5 | BP10 | BP20 | BP40 |
|---|---|---|---|---|---|
| Oct4 | 100% | 119% | 419% | 258% | 144% |
| SD | — | 0.001 | 0.283 | 0.129 | 0.008 |
| Sox2 | 100% | 91% | 245% | 210% | 140% |
| SD | — | 0.054 | 0.428 | 0.060 | 0.089 |

### Example 5

### Alkaline Phosphatase Staining

Alkaline phosphatase is a marker protein of stem cells. The activity measured by staining could be used to determine if BP can effectively maintain the self-renewal and/or pluripotency of stem cells.

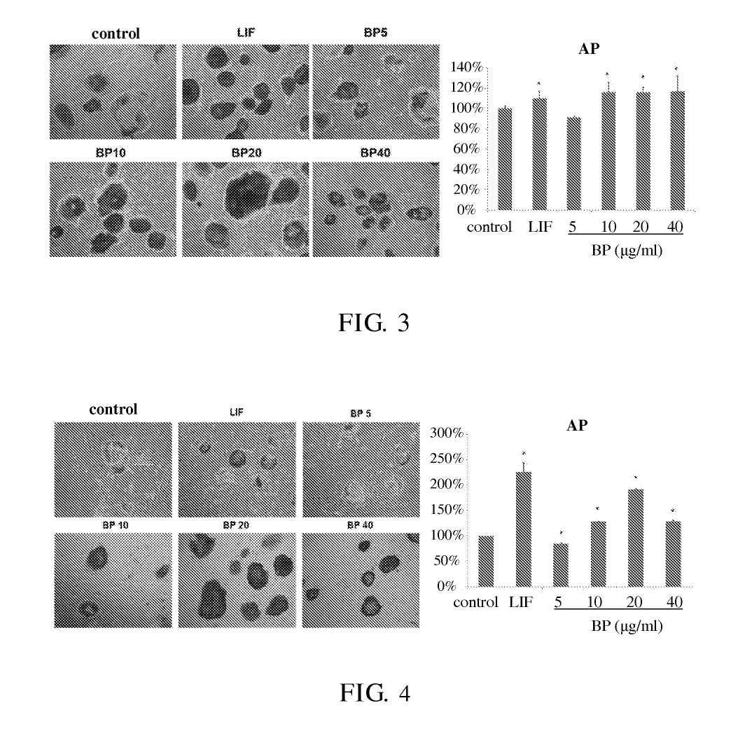

An experimental group medium (BP5, BP10, BP20 or BP40), a LIF-containing medium (without BP) and a control group medium (without BP) were used respectively to culture ES cells in a 6-well culture dish with an initial density of 1×10⁴ per well for 72 hours. The medium was removed and the cells were washed two times with PBS. Next, PBS was removed, and 1 ml of 80% ethanol was added into each well to perform the fixation at 4° C. for 2 to 24 hours. Next, ethanol was removed, and then the cells were washed one time with distilled water. The distilled water was added again to soak the cells for 2 to 3 minutes, and then be removed. Next, the cells were soaked with a Tris-HCl buffer (100 mM, pH 8.2 to 8.5) at room temperature for 5 minutes, and then, the buffer was removed. Next, a Leukocyte Alkaline Phosphatase kit (Vector) was used to perform the staining for 20 to 30 minutes, and then the Alkaline Phosphatase Substrate working solution was removed, the cells were soaked and washed by Tris-HCl buffer (100 mM, pH 8.2 to 8.5) and then be observed by a fluorescent microscope to count the number of the AP-positive clones. The undifferentiated ES cells with a higher activity show a red color, while the differentiated ES cells with a lower activity show a weak red color or are even colorless. The results are shown in FIG. 3 and Table 4.

By using the above culture conditions, an experimental group medium (BP5, BP10, BP20 or BP40), a LIF-containing medium (without BP), and a control group medium (without BP) were used respectively to culture iPS cells. Then, the staining of iPS cells was performed by the steps described above. The undifferentiated iPS cells with a higher activity show a red color and the differentiated iPS cells with a lower activity show a weak red color or are even colorless. The results are shown in FIG. 4 and Table 4.

TABLE 4

| | ES cells | | iPS cells | |
|---|---|---|---|---|
| | AP-positive clones | SD | AP-positive clones | SD |
| Control group | 100% | — | 100% | — |
| LIF | 110% | 0.074 | 226% | 0.179 |
| BP5 | 91% | 0.013 | 85% | 0.013 |
| BP10 | 116% | 0.104 | 128% | 0.002 |
| BP20 | 116% | 0.051 | 192% | 0.013 |
| BP40 | 117% | 0.152 | 129% | 0.018 |

As shown in FIG. 3, FIG. 4 and Table 4, the experimental group which comprises a BP-containing ES cell medium has more AP-positive clones as compared with the control group (without BP), and the number of AP-positive clones in the experimental group was equal to that of the group using LIF-containing medium (without BP). This result shows that BP indeed can maintain the self-renewal and pluripotency of ES and/or iPS cells.

### Example 6

### Immunofluorescence Staining

Nanog and SSEA1 are also marker proteins of stem cells, and their expression levels could be measured by immunofluorescence staining to further confirm whether BP can effectively maintain the self-renewal and pluripotency of stem cells.

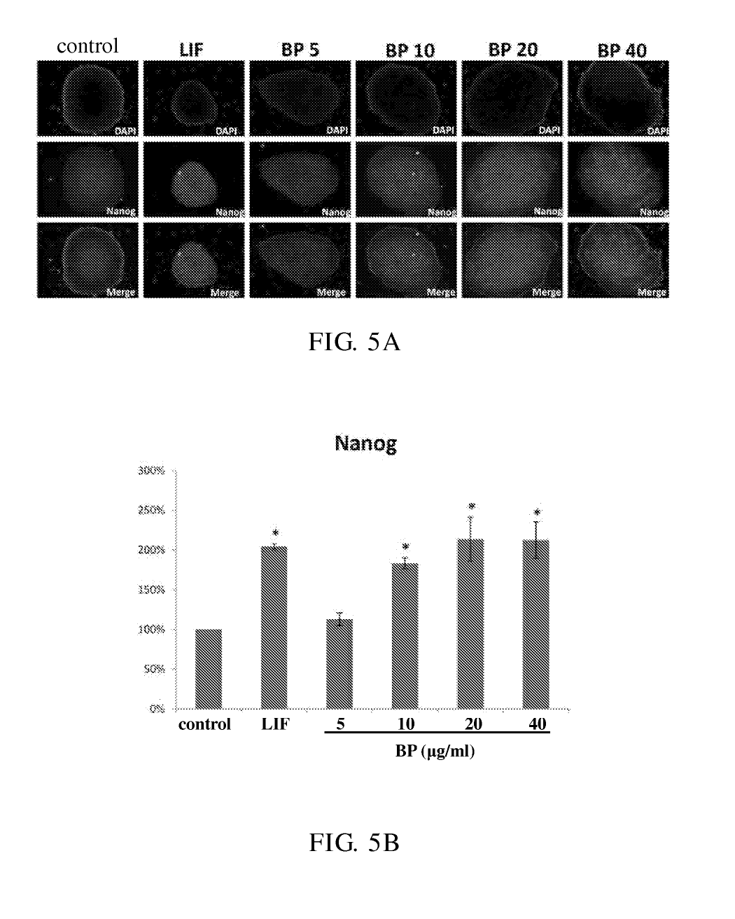

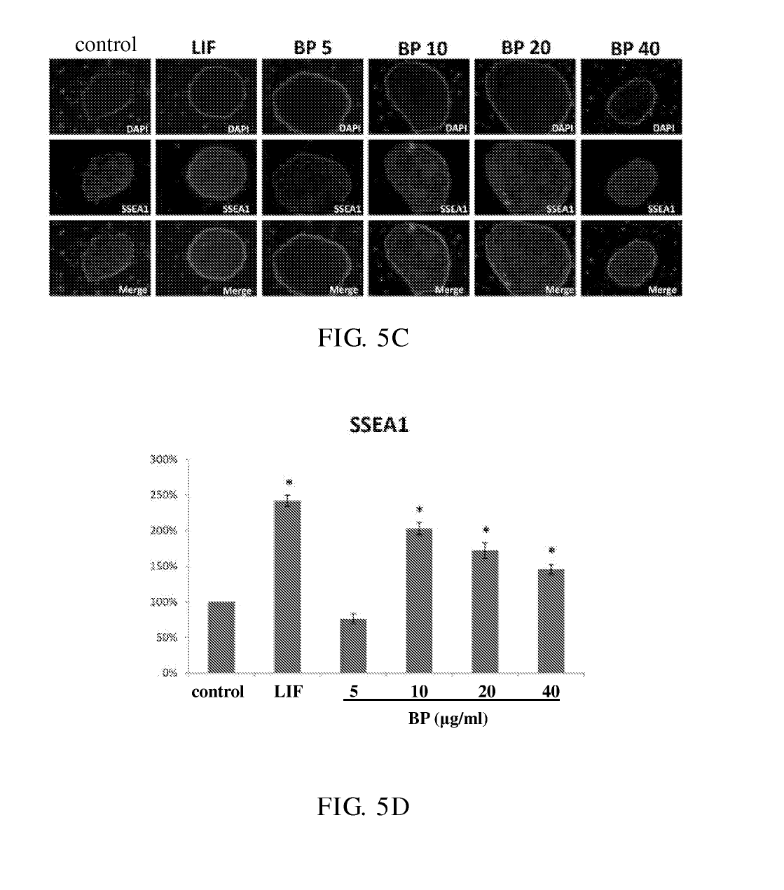

An experimental group medium (BP5, BP10, BP20 or BP40), a LIF-containing medium (without BP), and a control group medium (without BP) were used respectively in a 6-well culture dish which comprises a slide in each well to culture ES cells with an initial density of 1×10⁴ per well for 72 hours. The medium was removed and the cells were washed with PBS. Next, PBS was removed, and then 4% paraformaldehyde was added to perform the fixation at room temperature for 10 minutes. Next, paraformaldehyde was removed, and then slides were washed with 0.1% Tween-20/1X PBS three times for 10 minutes each time. The 0.3% Tween-20/1X PBS was added and held at room temperature for 30 minutes to penetrate the cell membrane to benefit the dye entry. After the slides were washed three times with 0.1% Tween-20, 5% FBS/1×PBS was added to perform a blocking reaction at room temperature for 2 hours, and then reacting with the primary antibody [anti-Nanog (Novus) or anti-SSEA1 (Millipore)] at a dilution of 1:100 at room temperature overnight. Slides were then washed 5 times with 0.1% Tween-20, then reacting with the secondary antibody [FITC-conjugated anti-mouse IgG or TRITC-conjugated anti-rabbit IgG (Sigma-Aldrich)] at a dilution of 1:500. After washed with 0.1% Tween-20 for 10 minutes, slides were mounted with the DAPI-containing UltraCruz™ Mounting medium and observed using an inverted fluorescent microscope. The results are shown in FIGS. 5A to 5D and Table 5.

TABLE 5

| | ES cells | | | |
|---|---|---|---|---|
| | Nanog | | SSEA1 | |
| | expression level | SD | expression level | SD |
| Control | 100% | — | 100% | — |
| group | | | | |
| LIF | 204.46% | 0.033 | 242.01% | 0.080 |
| BP5 | 113.22% | 0.078 | 76.27% | 0.070 |
| BP10 | 183.28% | 0.071 | 202.82% | 0.085 |
| BP20 | 213.8% | 0.277 | 172.12% | 0.111 |
| BP40 | 212.83% | 0.227 | 145.71% | 0.067 |

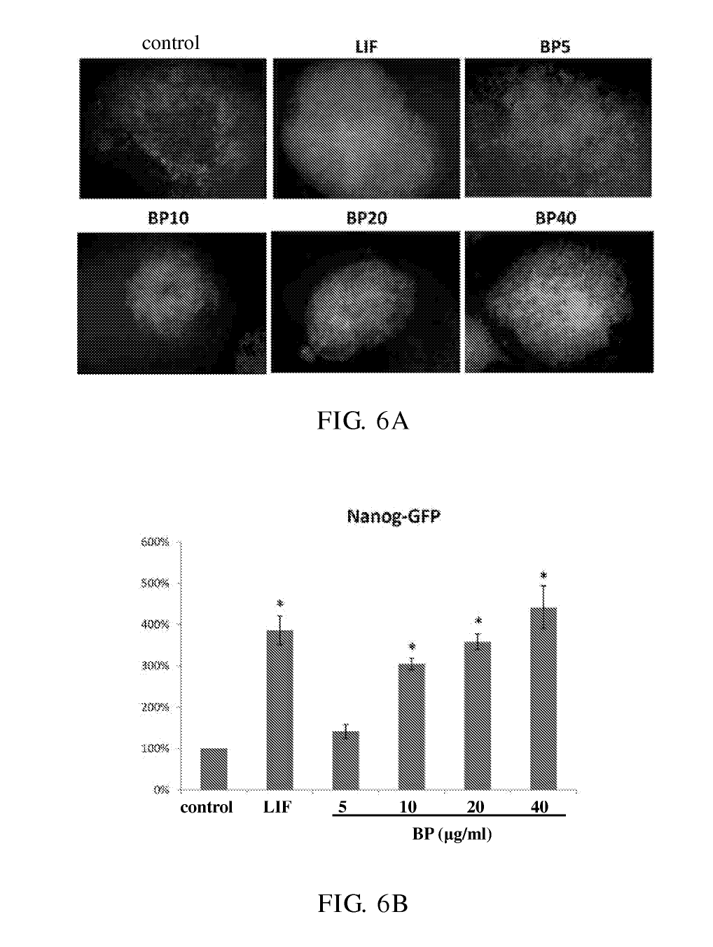

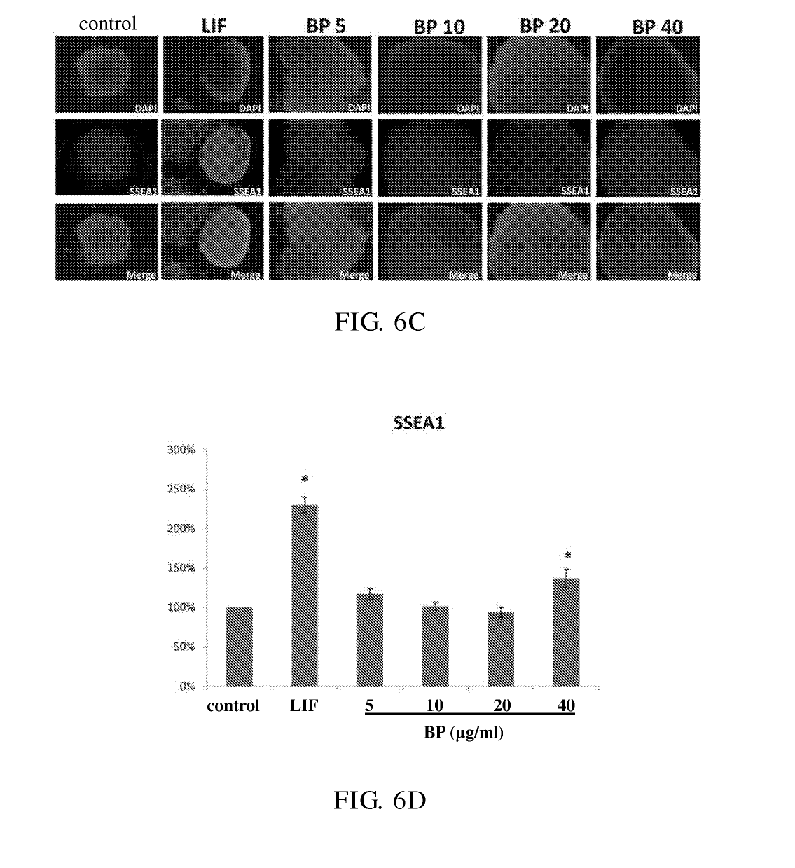

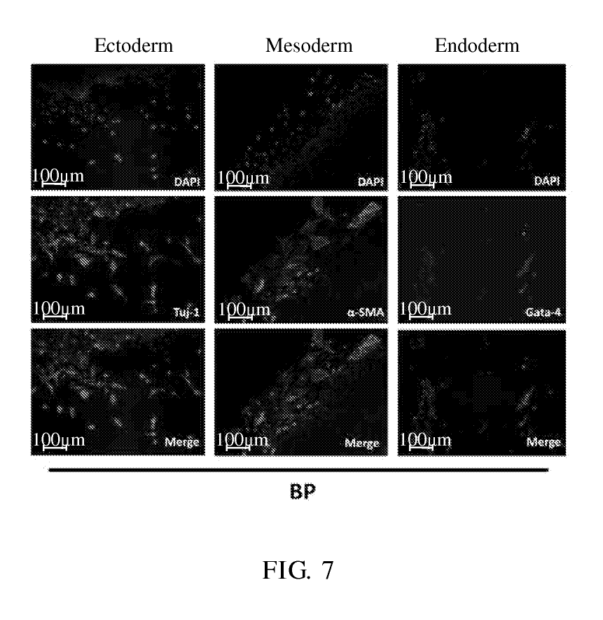

By using the above culture conditions, an experimental group medium (BP5, BP10, BP20 or BP40), a LIF-containing medium (without BP), and a control group medium (without BP) were used respectively to culture iPS cells. The staining of iPS cells was performed by using the steps described above. The results are shown in FIGS. 6A to 6D and Table 6.

TABLE 6

| | iPS cells | | | |
|---|---|---|---|---|
| | Nanog | | | |
| | expression level | SD | SSEA1 expression level | SD |
| Control | 100% | — | 100% | — |
| group | | | | |
| LIF | 386% | 0.350 | 230% | 0.099 |
| BP5 | 142% | 0.169 | 117% | 0.066 |
| BP10 | 305% | 0.134 | 101% | 0.046 |
| BP20 | 358% | 0.192 | 94% | 0.062 |
| BP40 | 442% | 0.521 | 137% | 0.119 |

As shown in FIGS. 5A to 5D, Table 5, FIGS. 6A to 6D and Table 6, the expression levels of Nanog and SSEA1 were higher in the experimental group which comprises a BP-containing ES cell medium as compared with the control group (without BP), and were equal to those of the group using LIF-containing medium (without BP). The foregoing result shows that BP indeed can maintain the self-renewal and pluripotency of ES and/or iPS cells.

### Example 7

### Embryoid Body Formation and Differentiation

The embryoid body formation and differentiation were also observed by immunofluorescence staining to observe the effect of BP on maintaining the pluripotency of ES cells.

An experimental group medium (BP10) was used to culture ES cells with an initial density of 1×10⁴ per well in a 6-well culture dish. The ES cells were passaged 3 times and incubated for another 2 to 3 days in the Ultra Low Cluster Plate (Costar) containing the embryoid body formation medium [DMEM+20% FBS+β-mercaptoethanol (1 mM)+1% L-glutamine+1% NEAA], and then the embryoid body formation was observed. The embryoid bodies were placed into another 24-well culture dish (5 to 6 embryoid bodies/well) and incubated for another 3 days. Next, immunofluorescence staining was conducted to detect cells with an anti-Tuj1 antibody (ectoderm marker), an anti-α-SMA antibody (mesoderm marker) and an anti-Gata4 antibody (endoderm marker). The results are shown in FIG. 7.

As shown in FIG. 7, the addition of BP into the ES cell medium can make an ES cell form an embryoid body and further differentiate into all three germ layer cells, including the endoderm, mesoderm and ectoderm. It was obvious that BP indeed can maintain the pluripotency of stem cells.

### Example 8

### Signaling Pathway in Stem Cells

Experiment A. DNA Microarray Analysis

The DNA microarray analysis was used to determine whether the gene expression profiles in various pathways in cells will be affected when the cells were cultured by a BP-containing medium.

An experimental group medium (BP10 or BP 40), a LIF-containing medium (without BP), and a control group medium (without BP) were used respectively in a 6-well culture dish to culture ES cells with an initial density of 1×10⁴ per well for 24 hours. Next, performing total RNA extraction and the obtained RNA was labeled with Cy3. Samples were hybridized to Agilent Mouse G3 Whole Genome Oligo 86×60K microarrays (Agilent) according to the manufacturer's instructions. Arrays were scanned with a Microarray Scanner System and data was analyzed by using a GeneSpring GX software (Agilent).

The biological function of genes and the signaling pathways that are involved were classified according to the KEGG and Babelomics databases to evaluate the numbers of genes significantly regulated in expression levels. The results are shown in Table 7. As shown in Table 7, the genes significantly regulated in the ES cells cultured with the BP-containing medium were majorly involved in the PPAR, ECM-receptor interaction, and/or Jak-Stat signaling pathways. According to the related research at present, the Jak-Stat signaling pathway is the most relevant in terms of stem cell self-renewal and pluripotency maintenance.

TABLE 7

| | BP10 | | | | BP40 | | | |
|---|---|---|---|---|---|---|---|---|
| Function/Pathway | I | D | No. | % | I | D | No. | % |

Signal transduction

| | | | | | | | | |
|---|---|---|---|---|---|---|---|---|
| PPAR signaling | 14 | 14 | 28/74 | 37.8 | 12 | 14 | 27/74 | 36.5 |
| pathway | | | | | | | | |
| ECM-receptor | 3 | 18 | 21/74 | 28.4 | 3 | 16 | 19/74 | 25.7 |
| interaction | | | | | | | | |
| JAK-STAT signaling | 9 | 30 | 39/140 | 27.9 | 8 | 27 | 35/140 | 25 |
| pathway | | | | | | | | |
| Calcium signaling | 4 | 28 | 32/146 | 21.9 | 2 | 30 | 32/146 | 21.9 |
| pathway | | | | | | | | |
| TGF-β signaling | 3 | 2 | 5/45 | 11.1 | 5 | 1 | 6/45 | 13.3 |
| pathway | | | | | | | | |
| MAPK signaling | 7 | 17 | 24/227 | 10.6 | 5 | 19 | 24/227 | 10.6 |
| Wnt signaling pathway | 2 | 5 | 7/66 | 10.6 | 0 | 6 | 6/66 | 9.1 |
| Insulin signaling | 3 | 7 | 10/102 | 9.8 | 2 | 3 | 5/102 | 4.9 |
| pathway | | | | | | | | |
| VEGF signaling | 2 | 0 | 2/31 | 6.5 | 0 | 0 | 0/31 | 0 |
| pathway | | | | | | | | |

Cell proliferation

| | | | | | | | | |
|---|---|---|---|---|---|---|---|---|
| Cell communication | 3 | 17 | 20/92 | 21.7 | 2 | 18 | 20/92 | 21.7 |
| Cell cycle | 0 | 4 | 4/163 | 2.5 | 0 | 6 | 6/163 | 3.7 |

Metabolism

| | | | | | | | | |
|---|---|---|---|---|---|---|---|---|
| Lipid metabolism | 3 | 15 | 18/109 | 16.5 | 2 | 12 | 10/109 | 12.8 |
| Amino acid | 0 | 1 | 1/23 | 4.3 | 0 | 2 | 2/23 | 8.7 |
| metabolism | | | | | | | | |

Cell adhesion

| | | | | | | | | |
|---|---|---|---|---|---|---|---|---|
| Cell adhesion | 13 | 20 | 33/150 | 22 | 13 | 21 | 34/150 | 22.7 |
| molecules | | | | | | | | |
| tight junction | 2 | 7 | 9/66 | 13.6 | 2 | 9 | 11/66 | 16.7 |
| focal adhesion | 3 | 16 | 19/173 | 11 | 2 | 20 | 22/173 | 12.7 |
| Apoptosis | 3 | 8 | 11/94 | 11.7 | 1 | 6 | 7/94 | 7.4 |

I: number of up-regulated genes;
D: number of down-regulated genes.

Experiment B. Real-Time Polymerase Chain Reaction (Real-Time PCR; Q-PCR)

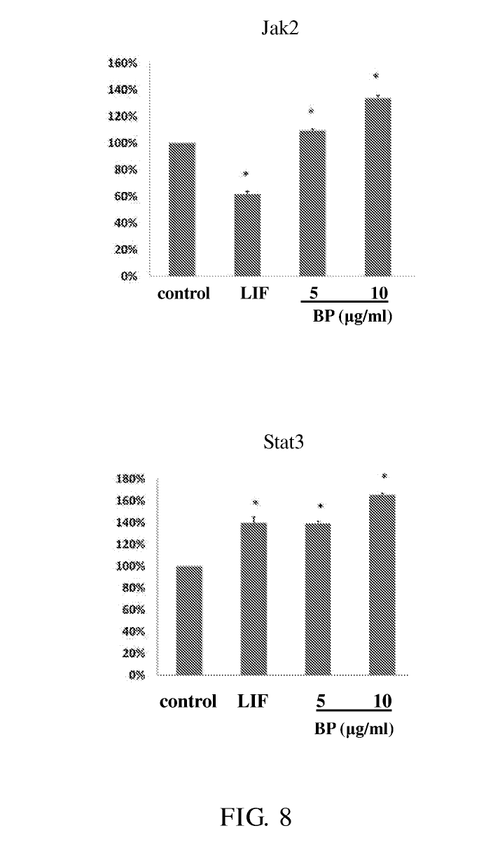

The protocol described in the Example 4 was repeated, an experimental group medium (BP5 or BP10), a LIF-containing medium (without BP), and a control group medium (without BP) were used respectively, while the real-time PCR was performed with the primers shown in Table 8 to detect the mRNA expression levels of Jak2 and Stat3 genes. The results are shown in FIG. 8. As shown in FIG. 8, the mRNA expression levels of Jak2 and Stat3 have been significantly up-regulated in cells cultured with the BP-containing ES cell medium.

TABLE 8

| | | Nucleic |
|---|---|---|
| | | acid sequence |
| Name | | of primer |
| Jak2 | Forward primer | CAATGATAAACAAGGGCAA |
| | (SEQ ID No: 5) | ATGAT |
| | Reverse primer | CTTGGCAATCTTCCGTTGCT |
| | (SEQ ID No: 6) | |
| Stat3 | Forward primer | CCCCGTACCTGAAGACCAAGT |
| | (SEQ ID No: 7) | |
| | Reverse primer | CCGTTATTTCCAAACTGCATCA |
| | (SEQ ID No: 8) | |
| LIF | Forward primer | CCTACCTGCGTCTTACTCCATCA |
| | (SEQ ID No: 9) | |
| | Reverse primer | TGTTTTCCCCAAAGGCTCAA |
| | (SEQ ID No: 10) | |
| EGF | Forward primer | GAGTCTGCCTGCGGATGGT |
| | (SEQ ID No: 11) | |
| | Reverse primer | GCTGCAGGGAGGGAGACA |
| | (SEQ ID No: 12) | |
| EPO | Forward primer | CCCCCACGCCTCATCTG |
| | (SEQ ID No: 13) | |
| | Reverse primer | TGCCTCCTTGGCCTCTAAGA |
| | (SEQ ID No: 14) | |
| IL5 | Forward primer | TCCCTGCTACTCTCCCCAAA |
| | (SEQ ID No: 15) | |
| | Reverse primer | CAACCTTCTCTCTCCCCAAGAA |
| | (SEQ ID No: 16) | |
| IL11 | Forward primer | CATGCCACACCCCAAACAA |
| | (SEQ ID No: 17) | |
| | Reverse primer | CCCCTCACCCAGGTCTACTG |
| | (SEQ ID No: 18) | |
| OSM | Forward primer | CGGTCCACTACAACACCAGATG |
| | (SEQ ID No: 19) | |
| | Reverse primer | GCGATGGTATCCCCAGAGAA |
| | (SEQ ID No: 20) | |

Experiment C. Western Blot Assay

According to the known research at present, Jak2 and Stat3 proteins could be phosphorylated to form the active forms, and the Jak2-Stat3 signaling pathway will be activated when Jak2 and Stat3 were phosphorylated.

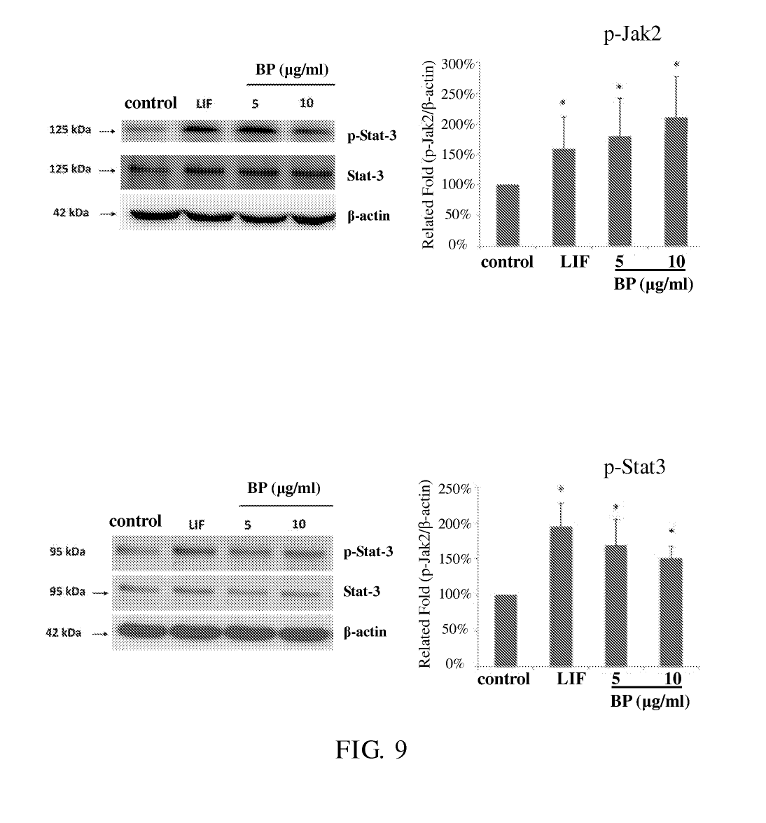

An experimental group medium (BP5 or BP10), a LIF-containing medium (without BP), and a control group medium (without BP) were used respectively to culture ES cells with an initial density of 1×10⁴ per well in a 6-well culture dish. The medium was removed until the cells growth to be about 70 to 80% confluence. The proteins were extracted to conduct the Western blot assay by using rabbit anti-Jak2 antibody (Cell Signaling Technology), mouse anti-phospho-Jak2 antibody (Cell Signaling Technology), rabbit anti-Stat3 antibody (BD) and rabbit anti-phospho-Stat3 antibody (Cell Signaling Technology), to determine whether BP would affect the protein expression levels of the genes in the Jak-Stat3 signaling pathway. The results are shown in FIG. 9 and Table 9.

As shown in FIG. 9 and Table 9, the total protein expression and the phosphorylated protein expression level of Jak2 and Stat3 increased significantly in the group cultured with the ES cell medium containing BP. This result explains that BP can maintain ES cell self-renewal by activating the Jak2-Stat3 signaling pathway.

TABLE 9

| | | Jak2 | SD | Stat3 | SD |
|---|---|---|---|---|---|
| Control group | Protein expression level | 100% | — | 100% | — |
| | Gene expression level | 100% | — | 100% | — |
| LIF | Protein expression level | 62% | 0.021 | 140% | 0.056 |
| | Gene expression level | 159% | 0.521 | 194% | 0.330 |
| BP5 | Protein expression level | 109% | 0.012 | 139% | 0.022 |
| | Gene expression level | 180% | 0.619 | 169% | 0.368 |
| BP10 | Protein expression level | 134% | 0.023 | 165% | 0.017 |
| | Gene expression level | 211% | 0.655 | 151% | 0.171 |

Experiment D. Examination of the Cytokine Genes Regulation

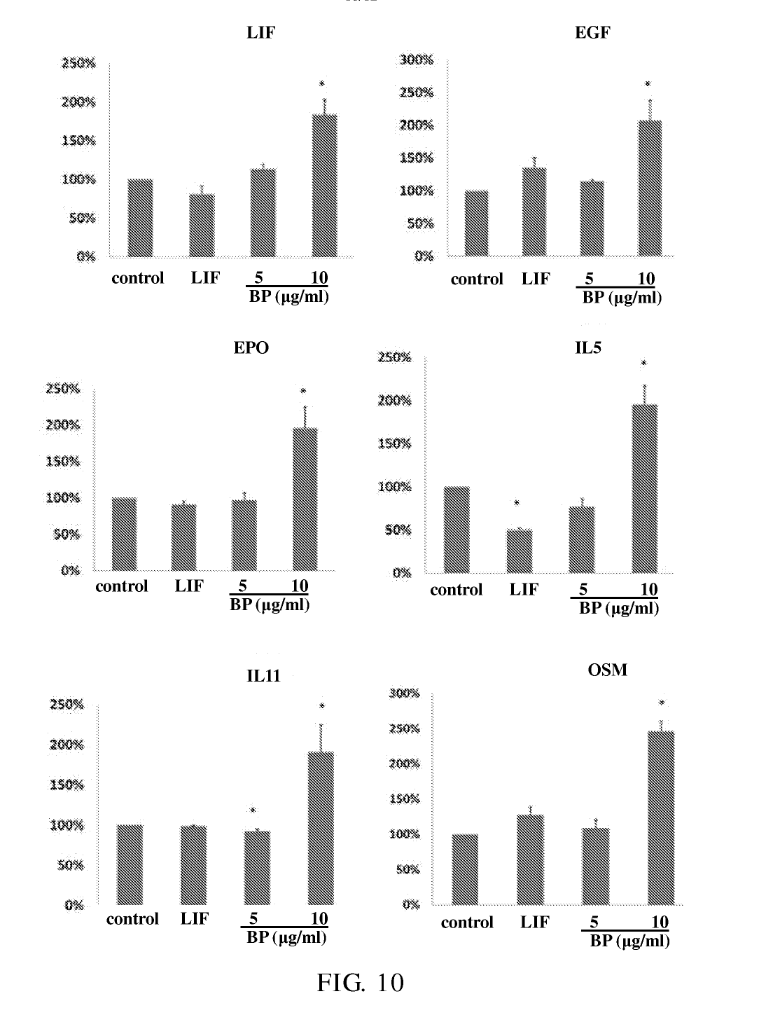

To understand how BP results in the activation of Jak2-Stat3 signaling, the protocol of Example 4 was repeated. An experimental group medium (BP5 or BP10), a LIF-containing medium, and a control medium were used respectively, while the real-time PCR was performed with the primers shown in Table 8 to detect the mRNA expression levels of Jak2 and Stat3 signaling pathway-related cytokine genes (LIF, EGF, EPO, IL-5, IL-11 and OSM). The results are shown in FIG. 10 and Table 10.

As shown in FIG. 10 and Table 10, the mRNA expression levels of the six genes have been significantly up-regulated in cells cultured with the BP-containing ES cell medium. The effect was most significant in the cells cultured with BP10. This result reveals that BP is capable of increasing Jak2-Stat3-related cytokine levels, thereby, activating Jak2 and Stat3 proteins to maintain stem cell pluripotency.

TABLE 10

| | Control group | LIF | BP5 | BP10 |
|---|---|---|---|---|
| LIF | 100% | 81% | 113% | 184% |
| SD | — | 0.111 | 0.073 | 0.190 |
| EGF | 100% | 145% | 110% | 204% |
| SD | — | 0.002 | 0.042 | 0.083 |
| EPO | 100% | 91% | 97% | 196% |
| SD | — | 0.044 | 0.102 | 0.289 |
| IL5 | 100% | 35% | 65% | 175% |
| SD | — | 0.016 | 0.083 | 0.017 |
| IL11 | 100% | 99% | 93% | 191% |
| SD | — | 0.008 | 0.021 | 0.342 |
| Osm | 100% | 128% | 109% | 246% |
| SD | — | 0.120 | 0.115 | 0.141 |

### Example 9

### Examination of iPS Cell Generation Efficiency

The MEF cells were isolated from Pou5fl-GFP transgenic mice (The Jackson lab). The pcDNA-Oct4, pcDNA-Sox2, pcDNA-c-Myc, and pcDNA-Klf4 plasmids were introduced once every two days (4 times total) into the Pou5fl-GFP MEF cells to generate iPS cells through co-transfection. The medium was changed to BP-containing medium 6 days post-transfection, and the medium was exchanged everyday. On day 9, the MEF cells were passaged onto feeder cells provided by Example 1 and the GFP-positive clones were observed. Finally, the GFP fluorescent signals of the iPS cells were detected by a fluorescent microscope. The fluorescent signal intensity is directly proportional to the cell generation efficiency.

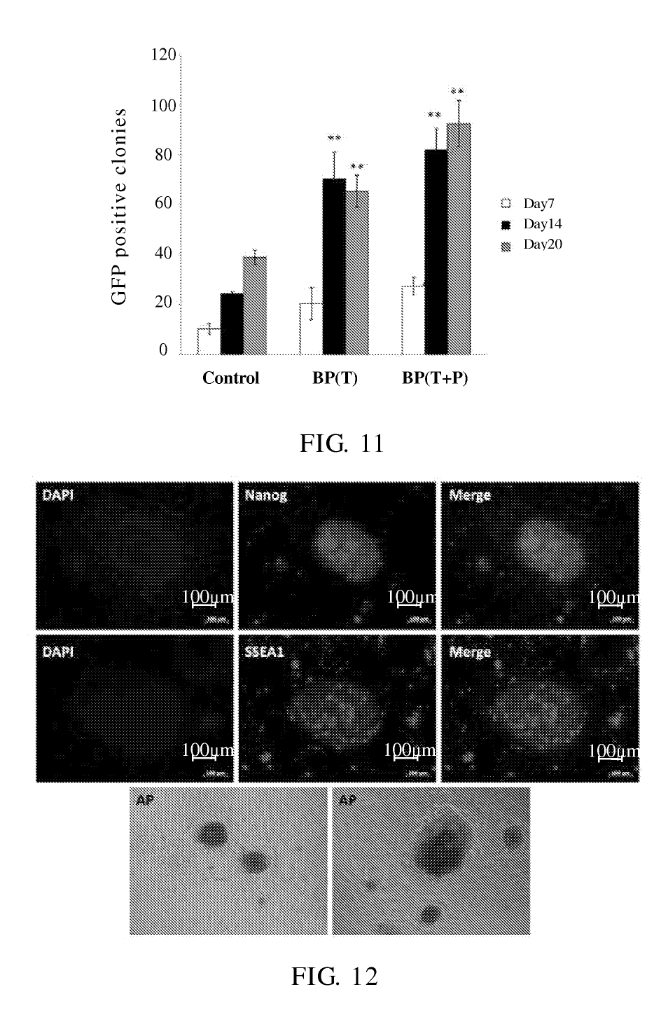

This example includes two groups, BP (T+P) and BP (T), wherein BP (T+P) refers to the group cultured with the experimental group medium (BP10) post-transfection and after placing onto feeder cells, and BP (T) refers to the group cultured with the experimental group medium (BP10) post-transfection. The results are shown in FIG. 11 and Table 11.

TABLE 11

| | Day 7 | Day 14 | Day 20 |
|---|---|---|---|
| Control group | 10.5 | 24.5 | 39 |
| SD | 2.121 | 0.707 | 2.828 |
| BP (T) | 20.5 | 70.5 | 65.5 |
| SD | 6.364 | 10.607 | 6.364 |
| BP (T + P) | 27.5 | 82 | 92.5 |
| SD | 3.536 | 8.485 | 9.192 |

As shown in FIG. 11 and Table 11, the experimental group which comprises a BP-containing iPS cell medium shows higher GFP fluorescent signals as compared with that of the control group (without BP). This result shows that BP indeed can enhance the generation efficiency of the iPS cell.

The AP activity of the obtained iPS cells was determined by AP staining. The expression levels of Nanog and SSEA1 as well as the embryoid body formation and differentiation were observed by immunofluorescence staining. The results are shown in FIG. 12. As shown in FIG. 12, the experimental group (T+P) which comprises a BP-containing iPS cell medium has higher expression levels of AP, Nanog, and SSEA1. This result shows that BP can not only enhance iPS cell generation efficiency but also maintain iPS cell self-renewal and pluripotency.

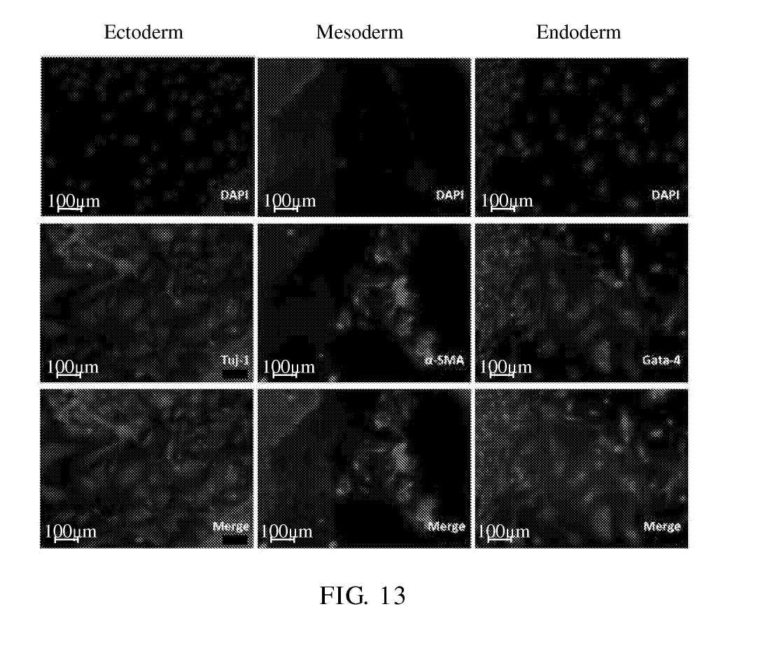

In addition, the iPS cells (T+P) were cultured by the steps shown in Example 7 to observe the embryoid body formation and differentiation. The results are shown in FIG. 13. As shown in FIG. 13, immunofluorescence staining was used to detect cells by using the anti-Tuj1 antibody (ectoderm marker), anti-α-SMA antibody (mesoderm marker), and anti-Gata4 antibody (endoderm marker). It was observed that the iPS cells can differentiate into three germ layer cells, i.e., endoderm, mesoderm and ectoderm. This result shows that BP could not only enhance the iPS cell generation efficiency but also further maintain the pluripotency iPS cell thus obtained.

According to the above results, the addition of BP into the stem cell medium in the present invention can not only enhance the stem cell generation efficiency but also maintain the pluripotency of the stem cell thus obtained during the culture procedure. Thus, the expensive LIF can be avoided, thereby, culturing stem cells in a economical way to improve the applicability of stem cells.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

1. A method for culturing a stem cell, comprising the following steps:
(A) providing a stem cell medium;
(B) adding purified n-butylidenephthalide (BP) into the stem cell medium to provide a BP-containing medium; and
(C) using the BP-containing medium to culture a stem cell.

2-20. (canceled)

21. The method as claimed in claim 1, wherein the stem cell is selected from the group consisting of an embryonic stem cell, a pluripotent stem cell, an induced pluripotent stem cell, a mesenchymal stem cell, an adipose stem cell, a hematopoietic stem cell, and combinations thereof.

22. The method as claimed in claim 1, wherein the stem cell is selected from the group consisting of an embryonic stem cell, a pluripotent stem cell, an induced pluripotent stem cell, and a combination thereof.

23. The method as claimed in claim 1, wherein the step (B) comprises:
(b1) dissolving purified BP into a solvent to form a BP-containing solution; and
(b2) mixing the BP-containing solution with the stem cell medium.

24. The method as claimed in claim 1, wherein the amount of purified BP used in the step (B) ranges from about 1 μg to about 80 μg per milliliter of the stem cell medium.

25. The method as claimed in claim 1, wherein the amount of purified BP used in the step (B) ranges from about 5 μg to about 50 μg per milliliter of the stem cell medium.

26. A method for culturing a stem cell, consisting of the following steps:
(A) providing a stem cell medium;
(B) adding n-butylidenephthalide (BP) into the stem cell medium to provide a BP-containing medium; and
(C) using the BP-containing medium to culture a stem cell.

27. The method as claimed in claim 7, wherein the stem cell is selected from the group consisting of an embryonic stem cell, a pluripotent stem cell, an induced pluripotent stem cell, a mesenchymal stem cell, an adipose stem cell, a hematopoietic stem cell, and combinations thereof.

28. The method as claimed in claim 7, wherein the stem cell is selected from the group consisting of an embryonic stem cell, a pluripotent stem cell, an induced pluripotent stem cell, and a combination thereof.

29. The method as claimed in claim 7, wherein the step (B) comprises:
(b1) dissolving BP into a solvent to form a BP-containing solution; and
(b2) mixing the BP-containing solution with the stem cell medium.

30. The method as claimed in claim 7, wherein the amount of BP used in the step (B) ranges from about 1 μg to about 80 μg per milliliter of the stem cell medium.

31. The method as claimed in claim 7, wherein the amount of BP used in the step (B) ranges from about 5 μg to about 50 μg per milliliter of the stem cell medium.


SEQUENCE LISTING (20 sequences)

SEQ ID NO: 1 - DNA, 21 bp, Artificial Sequence
  Oct4 primer- Forward Sequence
gaggctacagggacacctttc

SEQ ID NO: 2 - DNA, 18 bp, Artificial Sequence
  Oct4 primer- Reverse Sequence
gtgccaaagtggggacct

SEQ ID NO: 3 - DNA, 19 bp, Artificial Sequence
  Sox2 primer- Forward Sequence
agggctggactgcgaactg

SEQ ID NO: 4 - DNA, 19 bp, Artificial Sequence
  Sox2 primer- Reverse Sequence
tttgcacccctcccaattc

SEQ ID NO: 5 - DNA, 24 bp, Artificial Sequence
  Jak2 primer- Forward Sequence
caatgataaacaagggcaaatgat

SEQ ID NO: 6 - DNA, 20 bp, Artificial Sequence
  Jak2 primer- Reverse Sequence
cttggcaatcttccgttgct

SEQ ID NO: 7 - DNA, 21 bp, Artificial Sequence
  Stat3 primer- Forward Sequence
ccccgtacctgaagaccaagt

SEQ ID NO: 8 - DNA, 22 bp, Artificial Sequence
  Stat3 primer- Reverse Sequence
ccgttatttccaaactgcatca

SEQ ID NO: 9 - DNA, 23 bp, Artificial Sequence
  LIF primer- Forward Sequence
cctacctgcgtcttactccatca

SEQ ID NO: 10 - DNA, 20 bp, Artificial Sequence
  LIF primer- Reverse Sequence
tgttttccccaaaggctcaa

SEQ ID NO: 11 - DNA, 19 bp, Artificial Sequence
  EGF primer- Forward Sequence
gagtctgcctgcggatggt

SEQ ID NO: 12 - DNA, 18 bp, Artificial Sequence
  EGF primer- Reverse Sequence
gctgcagggagggagaca

SEQ ID NO: 13 - DNA, 17 bp, Artificial Sequence
  EPO primer- Forward Sequence
cccccacgcctcatctg

SEQ ID NO: 14 - DNA, 20 bp, Artificial Sequence
  EPO primer- Reverse Sequence
tgcctccttggcctctaaga

SEQ ID NO: 15 - DNA, 20 bp, Artificial Sequence
  IL5 primer- Forward Sequence
tccctgctactctccccaaa

SEQ ID NO: 16 - DNA, 22 bp, Artificial Sequence
  IL5 primer- Reverse Sequence
caaccttctctctccccaagaa

SEQ ID NO: 17 - DNA, 19 bp, Artificial Sequence
  IL11 primer- Forward Sequence
catgccacaccccaaacaa

SEQ ID NO: 18 - DNA, 20 bp, Artificial Sequence
  IL11 primer- Reverse Sequence
cccctcacccaggtctactg

SEQ ID NO: 19 - DNA, 22 bp, Artificial Sequence
  OSM primer- Forward Sequence
cggtccactacaacaccagatg

SEQ ID NO: 20 - DNA, 20 bp, Artificial Sequence
  OSM primer-  Reverse Sequence
gcgatggtatccccagagaa